(19) United States
(12) Patent Application Publication
(10) Pub. No.: US 2015/0010644 A1
(43) Pub. Date: Jan. 8, 2015

(54) OXYGENATION OF THE MUCOSAL BARRIER BY ENTERAL ARTIFICIAL BLOOD TO PREVENT HYPOXIC BREAKDOWN OF THE MUCOSAL BARRIER

(71) Applicant: THE REGENTS OF THE UNIVERSITY OF CALIFORNIA, Oakland, CA (US)

(72) Inventors: Geert W. Schmid-Schonbein, Del Mar, CA (US); Marisol Chang, Fort Worth, TX (US); Pedro Cabrales, San Diego, CA (US)

(73) Assignee: THE REGENTS OF THE UNIVERSITY OF CALIFORNIA, Oakland, CA (US)

(21) Appl. No.: 14/365,302

(22) PCT Filed: Dec. 14, 2012
(86) PCT No.: PCT/US2012/069956

§ 371 Date: Jun. 13, 2014

Related U.S. Application Data
(60) Provisional application No. 61/570,793, filed on Dec. 14, 2011.

(51) Int. Cl. A61K 33/00; A61K 47/06; A61K 47/26

(52) U.S. Cl. CPC A61K 33/00; A61K 47/26; A61K 47/06
USPC 424/600

(57) ABSTRACT

Techniques are disclosed that interfere with breakdown of the mucosal barrier in the stomach and the intestine under conditions of hypoxia by administrating a therapeutic dose of an oxygen rich solution. Such solution may include one or more of perflurocarbon, ATP solution or buffer solution.

This application claims priority to U.S. Provisional Patent Application Ser. No. 61/570,793, filed Dec. 14, 2011, the content of which is hereby incorporated by reference herein in its entirety into this disclosure.

### GOVERNMENT INTERESTS

This subject disclosure was made with U.S. Government support under Grant Nos. GM085072 and HL052684 awarded by the National Institute of Health (NIH). The government has certain rights in this subject disclosure.

### BACKGROUND OF THE SUBJECT DISCLOSURE

1. Field of the Subject Disclosure

The present subject disclosure relates to preservation of the mucosal barrier. In particular, the present subject disclosure relates to methods to treat or attenuate mucosal barrier damage.

2. Background of the Subject Disclosure

The intestinal mucosal barrier, composed of epithelium and a mucus layer, normally compartmentalizes pancreatic digestive enzymes in the lumen. However, during early periods of gut ischemia, trypsin and other enzymes appear in the wall of the small intestine initiating autodigestion.

There is currently no effective treatment to prevent breakdown of the mucosal barrier. Thus, there is a need for new methods for treatment of mucosal barrier breakdown. The methods should be simple to administer, effective and capable of aiding individuals in diminishing or preventing harmful effects of mucosal barrier breakdown without suffering from side effects.

### SUMMARY OF THE SUBJECT DISCLOSURE

The present subject disclosure provides techniques to interfere with breakdown of the mucosal barrier in the stomach and the intestine under conditions of hypoxia, and minimize escape of digestive enzymes and consequent destruction of tissue and generation of multi-organ failure. The approach is to administer an oxygen carrier directly into the lumen of the intestine and minimize oxygen depletion, enhance ATP production in the mucosal barrier, and thereby preserve epithelial mucosal barrier function.

### BRIEF DESCRIPTION OF THE DRAWINGS

FIG. 1 shows an in-situ zymography for trypsin in jejunal sections.

FIG. 2 shows representative micrographs of mucin2 and lectin fluorescent staining for SHAM and splanchnic arterial occlusion (SAO) groups with saline in the lumen and with perfluorocarbons with or without oxygen.

FIG. 3 shows Western blots for mucin2 and mucin13 and β-actin of intestine homogenates.

FIG. 4 shows representative micrographs of hypoxia staining with fluorescent antibody specific for pimonidazole hydrochloride for SHAM and SAO groups with saline in the lumen and with perfluorocarbons with or without oxygen.

FIG. 5 shows ATP concentrations in intestine homogenates.

FIG. 6 shows Thiobarbituric Acid Reactive Substances assay.

### DETAILED DESCRIPTION OF THE SUBJECT DISCLOSURE

There is currently no method to maintain oxygen at the mucosal barrier during elective (e.g., surgery that requires interruption of blood flow to the intestine like vascular reconstructions, intestinal lesion resections, tumor resections, etc.) or non-elective (due to trauma or disease related reductions of the blood flow to the intestine) clinical situations.

The general idea of the present subject disclosure is to deliver oxygen via an artificial oxygen carrier into the lumen of the intestine, either as preventive measure in anticipation of hypoxia in the intestine or to minimize tissue hypoxia as acute or chronic intervention.

In a typical application during surgery, an oxygen carrying solution, such as an artificial blood product able to carry oxygen (e.g., oxygen carrier Perfluorodecalin (C₁₀F18), CAS 306-94-5, 95% mixture of cis and trans isomerism, Perfluorocarbons), is saturated with oxygen and kept in an airtight container prior to surgery. The oxygen carrying solution can be administered into the lumen of the intestine orally, by, for example, nasogastric (NG) tube into the stomach, or by catheter into the duodenum before or during intestinal ischemia.

It has been shown in a rat model with severe ischemic intestine that enteral administration of an oxygen carrier serves to minimize intestinal damage by prevention of the entry of digestive enzymes into the wall of the intestine. The case was illustrated in a model with 30 min ischemia with Perfluorodecalin solution (7 ml for 230 g rat) saturated for 10 min in oxygen.

To investigate whether mucin provides a barrier against trypsin transport, IEC-18 cells (on Transwell plates) were coated with and without a mucin film (400 μm, 10%, porcine stomach). FITC-dextrans (20 kDa, 5 mg/ml) with and without trypsin (50 μM) was added to the apical side and the rate of FITC accumulation on the basal side over 60 min was measured. To determine if trypsin cleaves the adherent junctions of the epithelium, it was measured by Western blot the levels of intra- and extracellular domains of E-cadherin. The results indicate that without a mucin film, E-cadherin is cleaved and epithelial permeability is increased by 42% after incubation with trypsin. In contrast, when mucin is present, cleavage is reduced and permeability is decreased by 30% (p<0.05). These results suggest that in the absence of mucin in the mucus layer, such as occurs in ischemia, trypsin perturbs the epithelial layer by cleavage of E-cadherin and increases its permeability, resulting in transport of enzymes and macromolecules into the intestinal wall. Thus, mucin protects against trypsin-mediated increases in intestinal epithelial permeability.

The present findings have numerous uses, including but not limited to, treatment for prevention of sepsis, multi-organ failure and mortality, use in surgery in which blood flow to the intestine is intentionally or non-intentionally reduced, any form of intestinal complications associated with hypoxia from new born to the elderly.

Commercially, the present findings would be most applicable for groups, entities, or companies interested in shock, gastroenterological treatments, gas delivery in surgery, dialysis, and the like.

Further studies have confirmed the efficacy of the methodology used in the present subject disclosure, as discussed in further detail below.

During splanchnic ischemia, such as in surgery, trauma, shock, heart failure or thrombosis, blood flow and oxygen supply may be reduced or even stopped and may not meet the intestine's metabolic needs. The inventors determined that one characteristic of early periods of splanchnic ischemia is the degradation of the mucin glycoprotein covering the intestinal epithelium, making the intestinal wall accessible to lumenal pancreatic proteases. But the mechanism by which intestinal mucin is disrupted during ischemia is still uncertain.

Hypoxia and acidosis followed by ATP depletion and free radical production occur early during ischemic injury. These same factors have also been associated with disruption of mucin properties in-vitro. Studies on gastric mucin indicate that its rheological properties exhibit a pH-dependent sol-gel transition from a viscous polymer solution to a soft gel as pH is lowered below pH≈4. In the normal airway mucus hydration and thus its viscoelasticity is regulated by at least two signaling systems mediated by ATP and adenosine.

Different approaches have been developed to treat or ameliorate ischemic injury in the intestine. One opportunity is by supply of an extracellular source of oxygen to enterocytes, e.g. by perfusing the gut lumen with gaseous oxygen, oxygenated crystalloids or perfluorocarbon solutions. This approach ameliorates mucosal injury during ischemia and serves to maintain mucosal barrier function. Perfluorocarbons have a high oxygen delivery capacity, are nontoxic and biochemically inert, and capable of dissolving up to 40 percent oxygen by volume. The reversible oxygen solubility of perfluorocarbons has a potential therapeutic application in situations where tissue oxygen delivery is impaired, such as in intestinal ischemia. Since synthesis of ATP is a major rate limiting factor following adverse circulatory conditions encountered during ischemia, supplementation with ATP to raise tissue levels has been suggested as a treatment of shock and ischemia. ATP infusion before and during shock is protective if given prior to hemorrhage and improves the survival of animals.

Thus in this study the inventors determined whether the disruption of the mucin layer is due to ischemia-derived factors, such as hypoxia, ATP depletion, acidosis and by oxygen free radicals. Using a rat model of intestinal ischemia by splanchnic arterial occlusion (SAO) the inventors studied the fate of two mucin isoforms (mucin 2 and mucin 13) after lumenal supplementation with oxygenated perfluorcarbon, ATP-MgCl₂ or HEPES as a buffering solution.

Male Wistar rats (250-300 g, Harlan Sprague Dawley Inc, Indianapolis, Ind.) were randomly assigned to each group (n=4 per group). Rats were kept on solid food restriction for 12 hours prior to surgery with water ad libitum. After general anesthesia (Ketamine/Xylazine, 75 mg/kg BW/20 mg/kg BW, IM), splanchnic arterial occlusion (SAO) by clamping the superior mesenteric and celiac arteries was performed for 30 min. Sham groups were treated the same with the arteries separated but not ligated.

Control groups: Animals with saline injection into the lumen of the intestine, 3 ml/100 g Body Weight (BW), 30 min prior to SHAM or SAO surgery.

Perfluorocarbon groups: Perfluorodecalin (PFC) (Sigma-Aldrich, St Louis, Mo.) with oxygen, previously bubbled into the solution for 10 min, was injected into the lumen of the intestine 30 min prior SHAM or SAO surgery (SHAM+PFC+O₂, SAO+PFC+O₂). PFC without oxygen bubbled into the solution was injected 30 min prior SHAM or SAO surgery (SHAM+PFC, SAO+PFC).

ATP-MgCl₂ groups: ATP-MgCl₂ (Sigma-Aldrich), 25 mg/kg BW was injected in the lumen of the intestine 30 min prior to SHAM or SAO surgery (SHAM+ATP, SAO+ATP).

pH groups: Acidic saline (pH 5.5) injected in the lumen of the intestine 30 min prior SHAM surgery or HEPES buffer (20 mM) injected into the lumen of the intestine 30 min prior to SHAM or SAO surgery.

Free radicals groups: The hydroxyl radical scavenger, dimethylthiourea (DMTU, Sigma-Aldrich; 2.0 mg/g BW) was injected in the lumen of the intestine 30 min prior to SAO (SAO+DMTU).

After 30 min SAO or sham surgery, the animals were euthanized (Beuthanasia®; 0.22 ml/kg BW, IV).

Tissue cryosections: After euthanasia, jejunal sections (˜1 cm in length) were excised without removal of lumenal contents and suspended in Tissue-Tek O.C.T. Compound (Sakura Finetek, Torrance, Calif.), snap frozen in isopentane/liquid nitrogen, and stored at −80° C. for analysis. Cryosections (5 μm thickness) along the longitudinal axis of the villi were used throughout all experiments. Cryosections were fixed in 10% formalin solution and processed in a non-blinded fashion.

In-situ tissue zymography: In-situ zymography for trypsin activity in cryosections was assessed by measurement of fluorescence resulting from the proteolytic cleavage of the substrate (1 mM, Na-benzoyl-L-arginine-7-amido-methylcoumarin hydrochloride; Sigma-Aldrich) as described elsewhere with propidium iodine counterstaining (Sigma-Aldrich) (9). Slides were observed under an inverted microscope (20× and 60× objectives).

Mucin and lectin staining: The simultaneous visualization of lectins and mucin was carried out using Lectin GS-II from Griffonia simplicifolia, Alexa Fluor® 594 Conjugate (EnzChek®, Invitrogen, Carlsbad, Calif.) and specific antibody for mucin2 with FITC secondary antibody 1:10000 (Santa Cruz Biotechnology) and nuclei counterstaining with DAPI. Sections were observed on an inverted microscope (20× objective) using the appropriate fluorescent filters.

Hypoxia staining: In separate experiments tissue hypoxia was detected with Hypoxyprobe™-1 Kit (Natural Pharmacia International, Burlington, Mass., USA). This method utilizes a small molecular marker, pimonidazole, which after intravenous injection forms adducts with thiol containing proteins only in oxygen-starved cells. After general anesthesia as described above, pimonidazole-HCl (6 mg/100 g) was injected intravenously 30 min before SAO or SHAM; at 30 min the animal was euthanized and the jejuna section removed, embedded in O.C.T and frozen in liquid nitrogen. Then cryosections were immunostained using a rabbit antibody that binds to protein adducts of pimonidazole in hypoxic cells.

Homogenates of intestine and lumenal contents: Jejunal segments were excised without removal of lumenal contents. For enzyme activity measurements, segments of intestine were homogenized with CelLytic™ (Sigma-Aldrich) without addition of protease inhibitors. For Western blot assays the intestine segments were homogenized as above in the presence of protease inhibitors (5 mM EDTA, 5 mM N-Ethylmaleimide, 25 mM iodoacetamide, 5 mM benzamidine, 300 mM acarbose, 5 mM 6-aminocaproic acid, 1 mM protease inhibitor cocktail, (Sigma-Aldrich). In separate experiments the small intestine of sham animals was excised, and the lumenal contents were flushed with 20 ml saline. Homogenates or lumenal contents were centrifuged (16,000 g for 15 min at 4° C.), the supernatant was collected and protein concentration was assessed with the bicinchoninic acid protein assay (Thermo Scientific).

Western blot: 20 μg of tissue homogenate were separated by SDS-PAGE. Membranes were incubated with primary antibodies as follows: mucin2, mucin13 and trypsin 1:1000 (Santa Cruz Biotechnology), pancreatic amylase 1:1000 (GeneTex, San Antonio, Tex.), intra- and extra-cellular domains of E-cadherin and TLR4 1:1000 (Abcam), intracellular domain of TLR4 1:1000 (Invitrogen). Secondary antibodies were diluted 1:20000 (Santa Cruz Biotechnology) and detected with Super Signal West Pico (Thermo Scientific). The exposed x-ray films were scanned and label intensity was measured using digital gel analysis (NIH ImageJ software).

ATP assay: ATP concentrations in intestine homogenates were measured using ATP fluorometirc assay kit (BioVision, Mountain View, Calif.) according to manufacturer's protocol. Briefly jejuna sections were homogenized with perchloric acid (BioVision), centrifuged at 15,000×G for 5 min at 4° C. Samples were mixed with ATP reaction mix, incubated for 30 min protected from light and the fluorescence was measured with 535/587 (Ex/Em).

TBARS assay: Thiobarbituric Acid Reactive Substances (TBARS) was measured using the TBARS assay kit (Zeptometrix, Buffalo, N.Y.) according to manufacturer's protocol. Briefly jejuna sections were homogenized with perchloric acid (BioVision), centrifuged at 15,000×G for 5 min at 4° C. Samples were mixed with TBA reagent, incubated at 95° C. for 60 min, and cool down in ice bath for 10 min. Samples were centrifuged at 3000 rpm for 15 min and the supernatant were read at 530/550 (Ex/Em).

Statistical Analysis: Results are presented as mean±SEM. Unpaired comparisons of mean values between groups were carried out by one-way ANOVA followed by Bonferroni post-hoc comparisons. P<0.05 was considered significant.

Oxygenated PFC and DMTU Prevent Elevation of Trypsin Activity in the Intestinal Wall

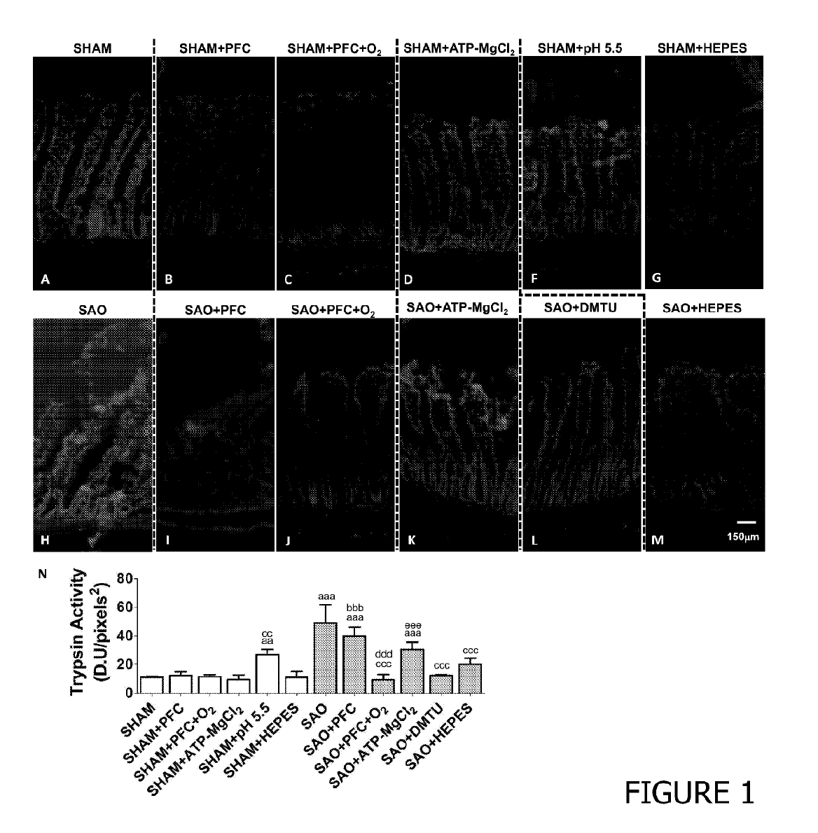

FIG. 1 shows in-situ zymography for trypsin in jejunal sections. (A-M) Representative micrographs of trypsin activity as observed by fluorescence of specific substrate (blue), nuclei counterstaining with propidum iodine (red) in SHAM animals or animals subjected to SAO protocol with luminal injection of perfluorocarbons with or without oxygen. (N) Enzymatic activity measured on the micrographs as mean fluorescent intensity of the fluorescent substrate; values are mean±SEM (n=4)/group. ^{aa}P<0.001, ^{aaa}P<0.0001 compared to SHAM, ^{bbb}P<0.0001 compared to SHAM+PFC, ^{cc}P<0.001, ^{ccc}P<0.0001 compared to SAO, ^{ddd}P<0.0001 compared to SAO+PFC, ^{eee}P<0.0001 compared to SHAM+ATP.

In-situ zymography revealed minimal trypsin activity in the intestinal wall of Sham groups (SHAM, SHAM+PFC, SHAM+PFC-O₂, SHAM+ATP, SHAM-HEPES) (FIG. 1A-D, G, N). High levels of trypsin activity were observed in the ischemic groups with either saline or deoxygenated perfluorocarbon in the lumen of the intestine (SAO and SAO+PFC) (FIG. 1H, I, N). Low levels of trypsin activity were seen in the ischemic groups with lumenal ATP-MgCl₂ (SAO+ATP-MgCl₂) and buffer solution (SAO-HEPES) (FIG. 1K, M, N) as well as in the Sham group with acidic saline (SHAM+pH 5.5) (FIG. 1F, N). Ischemic groups with lumenal injection of oxygenated perfluorcarbon or DMTU (SAO30+PFC+O₂, SAO+DMTU,) (FIG. 1J, L, N) had undetectable levels of trypsin activity in the intestinal wall.

Carbohydrate and Protein Portion of Mucin are Reduced During SAO

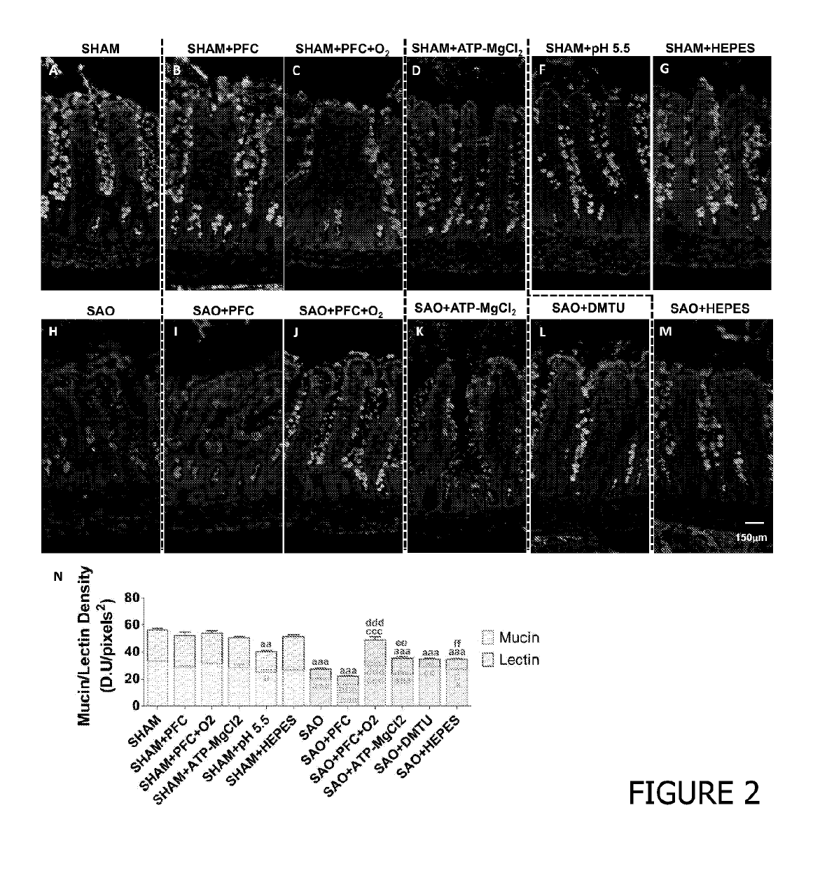

FIG. 2 shows mucin2 and lectin staining. (A-M) Representative micrographs of mucin2 and lectin fluorescent staining for SHAM and SAO groups with saline in the lumen and with perfluorocarbons with or without oxygen. (blue) nuclei, (red) lectin and (green) mucin2. (N) Mucin density measured as the mean light intensity after labeling with lectin or primary antibody against mucin2. Values are mean±SEM (n=4)/group; color symbols used: lectin (red), mucin2 (green). ^{aa}P<0.001, ^{aaa}P<0.0001 compared to SHAM, ^{bbb}P<0.0001 compared to SHAM+PFC. ^{cc}P<0.001, ^{ccc}P<0.0001 compared to SAO. ^{ddd}P<0.0001 compared to SAO+PFC. ^{eee}P<0.0001 compared to SHAM+ATP. ^{ff}P<0.001, ^{fff}P<0.0001 compared to SHAM+HEPES.

Double labeling of lectin and mucin2 reveals that the SAO group with saline in the lumen has lower levels of mucin2 and lectin staining as compared to SHAM with saline (FIG. 2A, H, N) especially at the interface between the villi tip and the intestinal lumen. The same trend was observed in the ischemic group with lumenal deoxygenated perfluorocarbon (SAO-PFC), which had significantly lower staining levels of both mucin2 and lectin as compared with SHAM and SHAM-PFC (FIG. 2B, I, N). Intralumenal supplementation with oxygenated perfluorocarbon in the ischemic group served to preserve the villus structure and both lectin and mucin2 staining were significantly higher as compared to SAO and SAO-PFC (FIG. 2C, J, and N). The SAO+DMTU group had undetectable villi injury during ischemia, no significant change in mucin2 staining levels but presented lower levels of lectin labeling as compared to SHAM (FIG. 2L, N). The ischemic group with lumenal ATP-MgCl₂ had mucin2 staining levels that were significantly lower as compared to the SHAM group with saline but was not statistically different from SHAM-ATP-MgCl₂. However, lectin staining was significantly lower as compared to the SHAM and SHAM-ATP-MgCl₂ (FIG. 2D, K, N) groups. The SHAM group with acidic saline (SHAM+pH5.5) presented lower levels of mucin2 and lectin staining as compared to SHAM (FIG. 2F, N). The ischemic group with lumenal HEPES buffer did not have signs of villi injury although mucin2 staining levels were significantly lower as compared to SHAM, and they were also significantly higher than SAO. In contrast, lectin levels were lower as compared to SHAM (FIG. 2G, M, N).

Mucin2 and Mucin13 Degradation is Mediated by Ischemic Factors

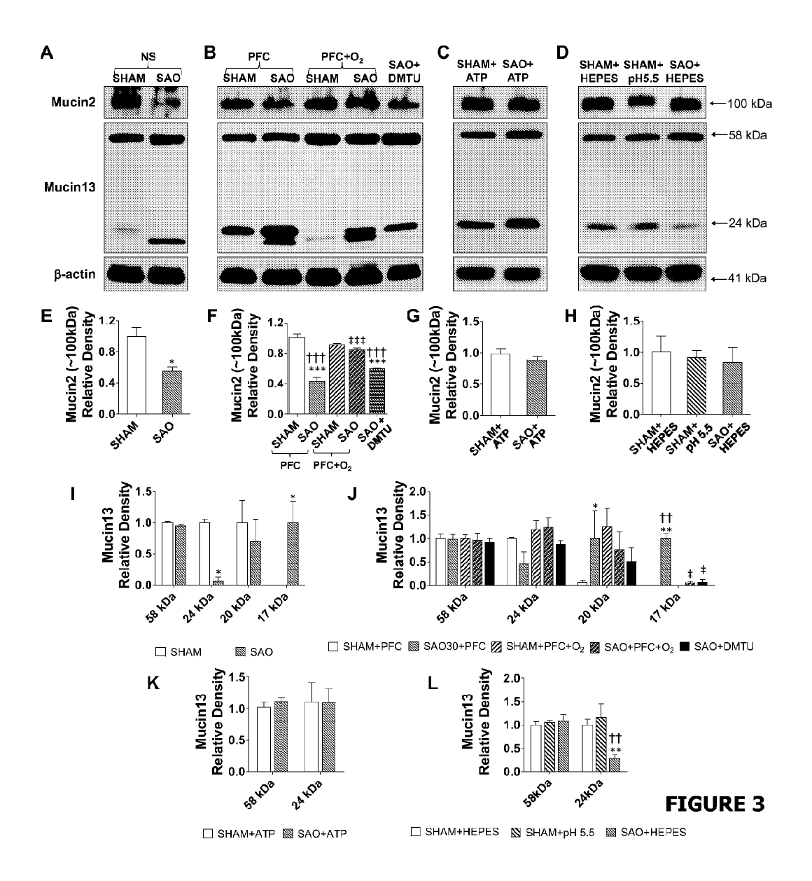

FIG. 3 shows Western blots for mucin2 and mucin13 and β-actin of intestine homogenates of (A) SHAM and SAO animals with normal saline in the lumen of the intestine. (B) SHAM and SAO animals with perfluorocarbon with or without oxygen and SAO with DMTU treatment in the lumen of the intestine. (C) SHAM and SAO animals with ATP-MgCl₂ in saline in the lumen of the intestine. (D) SHAM+HEPES and SHAM+pH5.5 and SAO+HEPES in the lumen of the intestine. (E and I) Relative densities of mucin2 and mucin13 with respect to β-actin Values are mean±SEM (n=4)/group (E and I) *P<0.05 compared to SHAM. (F and J) *P<0.05 and **P<0.001 compared to SHAM+PFC, ††P<0.001 compared to SHAM+PFC+O2, ‡P<0.05 and ‡‡P<0.001 compared to SAO+PFC. (H and L) **P<0.001 compared to SHAM+HEPES, ††P<0.001 compared to SHAM+pH5.5.

Western blots of mucin2 and mucin13 for sham and ischemic groups with lumenal saline (SHAM, SAO) indicate that mucin2 relative density significantly decreases after ischemia (FIGS. 3A, E). Mucin13 western blot show that in the SHAM group there are low molecular weight bands around 24 kDa, however after ischemia those bands are no detected or present in less quantities than SHAM with the appearance of a lower molecular weight band around 17 kDa that was not present in the SHAM group (FIG. 3A, I). Injection of deoxygenated perfluorcarbon in sham and ischemic groups (SHAM+PFC, SAO+PFC) presented similar results are those observed with groups with only saline injection. Mucin2 protein levels were reduced in the SAO group (SAO+PFC) as compared to sham (SHAM+PFC) (FIG. 3B, F). Mucin13 levels also presented the lower molecular weight fragments at 17 kDa with the only difference that bands around 24 kDa did not completely disappear in the ischemic group (SAO+PFC) as compared to the group with just saline (SAO) (FIG. 3B, F). Injection of oxygenated perfluorocarbon in the ischemic group (SAO+PFC+O₂) resulted in mucin2 density levels similar to the sham (SHAM+PFC+O₂) (FIG. 3B, F). Mucin13 levels in this group resulted with complete disappearance or very faint levels of the 17 kDa band in the ischemic group (SAO+PFC+O₂) as compared to sham (SHAM+PFC+O₂) (FIG. 3B, J). Lumenal injection of DMTU in the ischemic group (SAO+DMTU) resulted in reduced levels of mucin2 but mucin13 western blot reveal no appearance of the lower molecular weight band at 17 kDA (FIG. 3B, F, J). This was also observed in the groups with ATP-MgCl₂ injection in the lumen of the intestine where the ischemic group (SAO+ATP-MgCl₂) have the same mucin2 and mucin13 protein levels as the sham (SHAM+ATP-MgCl₂) (FIG. 3C, G, K). The same pattern was observed for the groups with either HEPES buffer of acidic saline. The sham group with acidic saline (SHAM+pH5.5) and ischemic group with HEPES buffer (SAO+HEPES) had similar levels of mucin2 and mucin13 as compared to sham (SHAM+HEPES).

Oxygenated Perfluorocarbon Prevents Intestinal Hypoxia

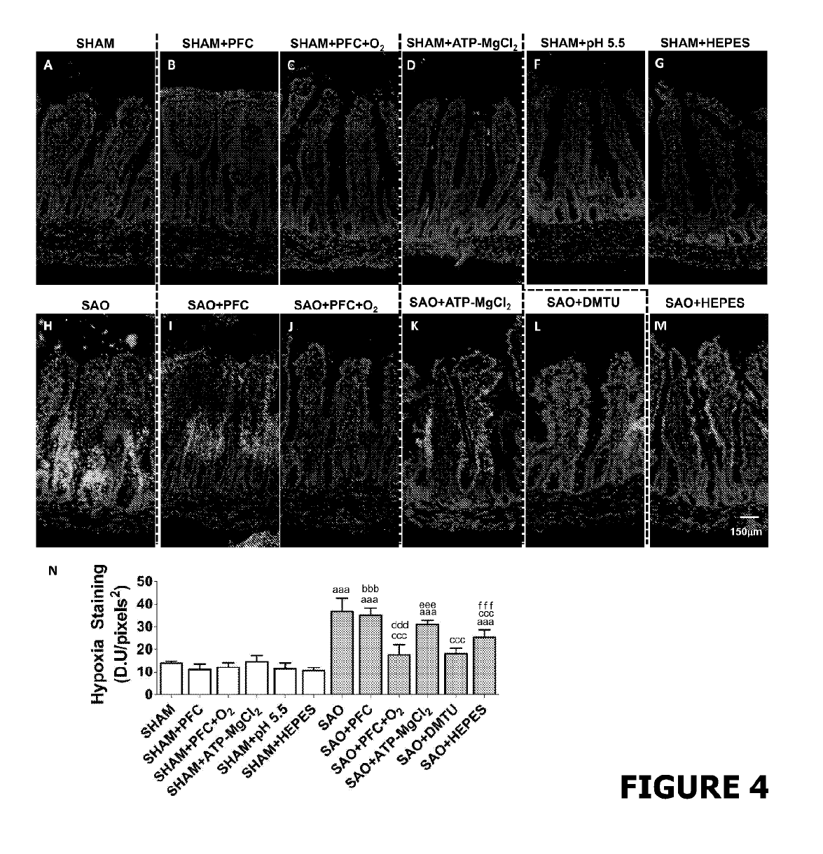

FIG. 4 shows hypoxia staining. (A-M) Representative micrographs of hypoxia staining with fluorescent antibody specific for pimonidazole hydrochloride for SHAM and SAO groups with saline in the lumen and with perfluorocarbons with or without oxygen. (blue) nuclei, (red) and (green) pimonidazole hydrochloride. (N) Hypoxia staining density measured as the mean light intensity after labeling with primary antibody against pimonidazole. Values are mean±SEM (n=4)/group. ^{aaa}P<0.0001 compared to SHAM, ^{bbb}P<0.0001 compared to SHAM+PFC. ^{ccc}P<0.0001 compared to SAO. ^{ddd}P<0.0001 compared to SAO+PFC. ^{eee}P<0.0001 compared to SHAM+ATP. ^{fff}P<0.0001 compared to SHAM+HEPES.

Hypoxia staining demonstrates that all sham operated animal had low levels of the hypoxia staining in the intestinal villi (FIG. 4A-G, N). Groups that underwent 30 min ischemia with luminal injection of saline and deoxygenated perfluorocarbon (SAO and SAO+PFC) had the highest levels of hypoxia staining (FIG. 4H, I, N). Luminal injection with oxygenated perfluorocarbon (SAO-PFC-O₂) resulted in hypoxia staining levels similar to the ones observed in the SHAM, SHAM-PFC and SHAM-PFC-O₂ groups, which was significantly lower as compared to SAO and SAO-PFC groups (FIGS. 4J and N). Lumenal injection of ATP-MgCl₂ in the ischemic group also presented significantly high levels of hypoxia staining as compared to SHAM groups (FIGS. 4K, and N). Lumenal injection with DMTU in the ischemic group resulted in significantly low levels of hypoxia staining as compared to SAO (FIGS. 4L, and N). The ischemic group with luminal injection of HEPES buffer had hypoxia staining levels that were significantly higher than the SHAM and SHAM-HEPES groups as well as the SAO group (FIGS. 4M and N).

ATP Concentration in Intestine Homogenates Decreases During SAO

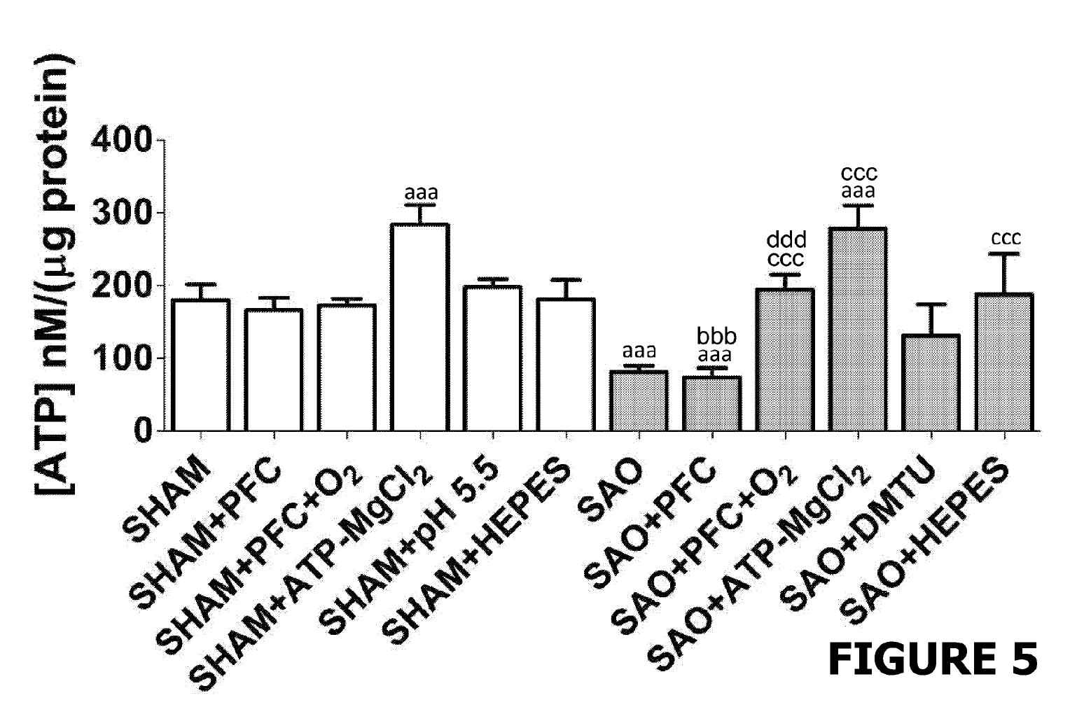

FIG. 5 shows ATP concentrations in intestine homogenates. Values are mean±SEM (n=4)/group expressed as [ATP] per pg of protein. ^{aaa}P<0.0001 compared to SHAM, ^{bbb}P<0.0001 compared to SHAM+PFC, ^{ccc}P<0.0001 compared to SAO, ^{ddd}P<0.0001 compared to SAO+PFC.

The concentration of ATP in intestinal tissue homogenates after 30 min ischemia significantly decreased in the groups with lumenal saline and deoxygenated perfluorocarbon (SAO, SAO+PFC) as compared to SHAM or SHAM+PFC (FIG. 5). The ischemic group with lumenal injection of oxygenated perfluorocarbon (SAO+PFC+O2) presented no reduction of ATP concentration as compared to SAO or SAO+PFC. Lumenal Injection of ATP-MgCl₂ significantly increased the ATP concentration in both sham and ischemic groups (SHAM+ATP-MgCl₂ and SAO+ATP-MgCl₂) as compared to those groups with only luminal saline (SHAM and SAO). The ATP levels in the ischemic intestine with luminal DMTU (SAO+DMTU) was lower than SHAM and higher than SAO but it was not statistically significant (FIG. 5). The ischemic group with HEPES buffer (SAO+HEPES) had ATP levels that were significantly higher than SAO and similar to sham groups (SHAM and SHAM+HEPES, FIG. 5)

Oxygenated Perfluorocarbon Reduces Levels of Thiobarbituric Acid Reactive (TBAR) Substances

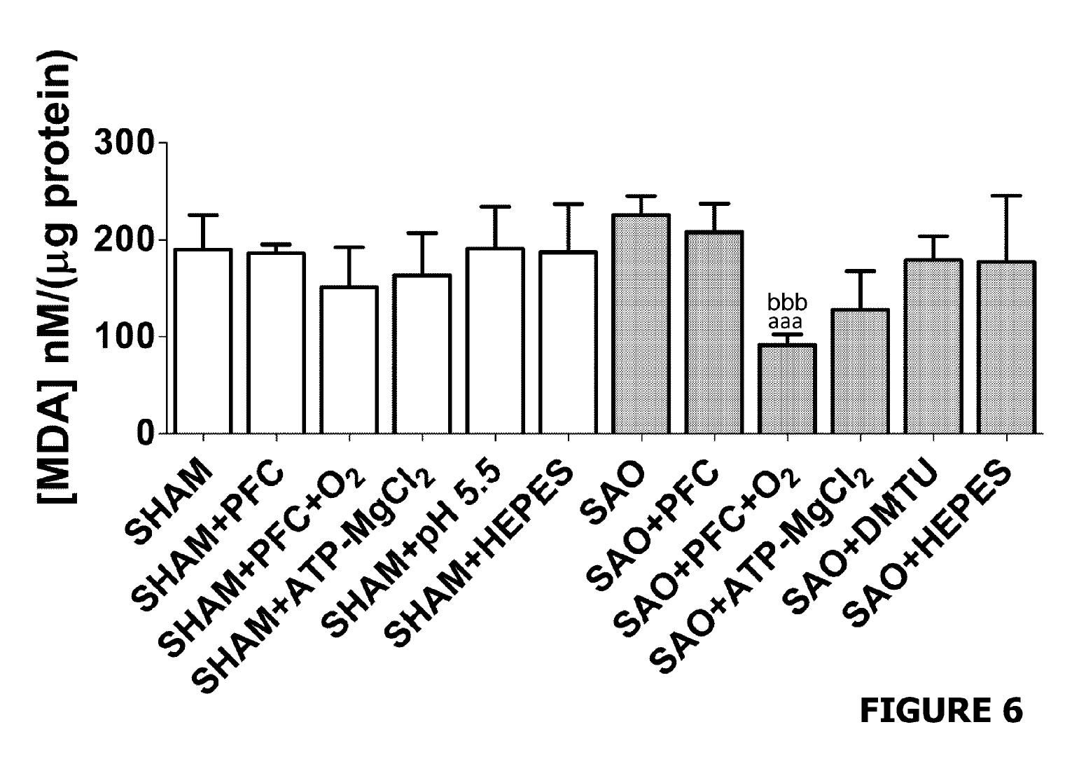

FIG. 6 shows thiobarbituric Acid Reactive Substances assay. Values are mean±SEM (n=4)/group expressed as [MDA] equivalent per μg of protein. ^{aaa}P<0.0001 compared to SAO, ^{bbb}P<0.0001 to SAO+PFC.

There were no differences between groups in the sham animals (FIG. 6). Lumenal injection with oxygenated perfluorocarbon resulted in lower levels of MDA equivalents in the ischemic group (SAO+PFC+O₂) (FIG. 6); and these levels were significantly lower as compared to SAO or SAO+PFC. The SAO group with lumenal ATP-MgCl₂ injection had on average lower levels of MDA equivalents compared to SHAM or SAO, but they were not significant. There was no significant changes in the average MDA levels in the other groups as compared to SHAM or SAO (FIG. 6).

The inventors have previously shown that mucin2 and mucin13 molecules are disrupted after intestinal ischemia facilitating the entry of digestive enzymes from the lumen into the wall of the intestine. The current results support the hypothesis that hypoxia is directly involved in the mucin degradation. Supplementation of oxygenated perfluorocarbon inside the lumen of the intestine prior to intestinal ischemia reduces mucin2 and mucin13 degradation and prevents the appearance of trypsin activity inside the intestinal wall.

Although the exact mechanism by which hypoxia results in degradation of mucin is unknown, the current data suggests an involvement by oxygen free radicals, specifically the hydroxyl radical. Reactive oxygen species (ROS) play an important role in the pathogenesis of the gastrointestinal track and ischemia-induced intestinal damage. The hydroxyl radical can damage virtually all types of macromolecules including carbohydrates, nucleic acids, lipids and amino acids. It has been reported that gastrointestinal mucin may function as free radical scavenger due to their high concentration of carbohydrate moieties. In-vitro incubation of mucin with hydroxyl radicals results in drop in mucin viscosity, suggesting possible damage to the molecule. Lectin staining indicates that the carbohydrate portion of mucin is significantly decreased in all SAO groups with the exception of the group that had oxygen supplementation with the perfluorcarbon (FIG. 2). Although the MDA measurements indicate there may be no significant increase of ROS in the ischemic group with luminal saline, oxygen and ATP supplementation decreased MDA levels. This may suggest that the shift in oxygen and ATP levels during ischemia may be directly involved in the mucin's ability to scavenge free radicals. Furthermore, It has been previously shown that MUC3, a transmembrane mucin, is selectively responsive to hypoxia via a (hypoxia-inducing factor 1) (HIF-1) transcription-dependent pathways, which results in elevated MUC3 mRNA levels. HIF-1 exists as an αβ heterodimer, the activation of which depends on stabilization of an O₂-dependent degradation domain of the α subunit by the ubiquitin-proteasome pathway (33). Similarly, we have previously shown that intestinal ischemia results in elevated levels of MUC2 mRNA (7) which may occur via a similar mechanism as in hypoxia induced MUC3 expression. Elevated mucin mRNA levels as a result of hypoxia may indicate enhanced replenishment of the disrupted mucin molecules (REF).

The role of hypoxia and ROS was confirmed with dimethylthiourea (DMTU), a hydroxyl radical scavenger that is able to penetrate into the intracellular space. It prevented mucin disruption. DMTU injection in the lumen of the intestine prior to ischemia (SAO+DMTU) results in protection of the intestine, reduced trypsin activity in the intestinal wall. The inventors also observed preservation of mucin13 protein levels but a reduction in mucin2 protein levels. Double labeling of lectin and mucin2 show decrease in lectin staining but not mucin2, which indicate that free radicals may be involved in the disruption of mucin2 protein core but not its carbohydrates. On the other hand, hypoxia seems to directly mediate the disruption of both the protein and carbohydrate portions of mucin.

Mucus hydration and its viscoelasticity in the airway mucosa is regulated by ATP and adenosine signaling (11). Since ATP tissue levels decrease during ischemia and there is increase intestinal permeability, ATP may be indirectly affecting mucin properties and subsequently increase intestinal permeability. The ischemic groups with ATP supplementation present less trypsin activity in the wall as compared to the ischemic group with only lumenal saline. There is also preservation of mucin2 staining as well as protein levels of mucin2 and mucin13. Therefore these results suggest that ATP depletion may be a player responsible of the degradation of mucin although the exact mechanism remains to be identified. There are several possible pathways by which ATP may mediate mucin homeostasis. For instance there is feedback loop involving surface pH and ATP concentration mediated by purinergic receptor. Also ATP may regulate mucin hydration by transmembrane conductance regulator (CFTR) or ENAC channels.

Mucin molecules in solution can cross-link to form aggregates via H-bonds, electrostatic and hydrophobic interactions, as well as Van der Waals forces. This cross-linking leads to the formation of a gel network that can be affected by disulfide bonds between lectins, mucin hydration, ionic strength or pH which can cause conformational changes from an isotropic random coil to an anisotropic extended random coil. A reduction in pH from 7.4 to 6.5 produces a significant decrease in the velocity of mucin swelling from the secretory granule matrix. However, when HCl was injected into solutions of gastric mucin, viscous channels are formed dependent on pH and mucin concentration, providing a possible explanation for the transport of acid through the lumen of the stomach. It has been proposed that among the functions of the mucin-bound carbohydrates include protease resistance, large water-holding capacity and high charge density from sialic acid and sulfate residues, which are charged at neutral pH. A change of lumenal pH in the lumen of the intestine changes the charge of the mucin molecule thus affecting mucin hydration. The results presented in this study show that when acidic saline (pH 5.5) was injected into the lumen of a sham animal there was a slight increase in trypsin activity in the wall of the intestine and a non-significant decrease in mucin2 density as compared to sham group with HEPES buffer, mucin13 molecule appeared unaffected by addition of acidic saline. A possible explanation for this behavior is that in the lumen of the sham intestine the acidic saline was buffered by the normal mechanisms thus preventing major injury, other experiments are needed in which acidic conditions in the lumen of the intestine are sustained. When HEPES buffer was added in the lumen of the intestine (SAO+HEPES) there was no significant decrease in mucin2 molecule as compared to sham (SHAM+HEPES) and there was no degradation of mucin13 which suggests that acidic pH does play a role in mucin degradation.

In conclusion, the results presented here confirm that during ischemia mucin isoforms are degraded, facilitating the entry of digestive enzymes into the wall of the intestine. The possible sequence of events is that during intestinal ischemia and hypoxia a depletion of ATP and lumenal acidosis arises. These events in combination alter the mucin structure making the molecule susceptible to enzymatic degradation. Treatment of ischemic factors with oxygenated perfluorocarbon, ATP-MgCl₂ supplementation or a buffering agent such as HEPES results in protection of mucin with reduced escape of digestive enzymes from the lumen of the intestine thus attenuating intestinal injury.

The following references, some whose findings or techniques are discussed or cited above, are hereby incorporated by reference herein in their entirety into this disclosure, as examples of using the present subject disclosure, and should be considered as part of this subject disclosure:
Chang, M. (2012). “Disruption of the Mucosal Barrier During Gut Ischemia Allows Entry of Digestive Enzymes into the Intestinal Wall.” Shock, 37(3), 297-305.
Chang, M. (2012). “Breakdown of Mucin as Barrier to Digestive Enzymes in the Ischemic Rat Small Intestine”. PloS ONE, 7(6), 1-12.
Schmid-Schonbein, G. W. (2011). “The Autodigestion Hypothesis in Shock and Multi-Organ Failure: Degrading Protease Activity”. Boletim da SPHM, 26(3), 6-15.

The foregoing disclosure of the preferred embodiments of the present subject disclosure has been presented for purposes of illustration and description. It is not intended to be exhaustive or to limit the subject disclosure to the precise forms disclosed. Many variations and modifications of the embodiments described herein will be apparent to one of ordinary skill in the art in light of the above disclosure. The scope of the subject disclosure is to be defined only by the claims appended hereto, and by their equivalents.

Further, in describing representative embodiments of the present subject disclosure, the specification may have presented the method and/or process of the present subject disclosure as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present subject disclosure should not be limited to the performance of their steps in the order written, and one skilled in the art can readily appreciate that the sequences may be varied and still remain within the spirit and scope of the present subject disclosure.

What is claimed is:

1. A method for treating hypoxic breakdown of a mucosal barrier, the method comprising:
administering to an individual a therapeutic dose of an oxygen carrying solution.

2. The method of claim 1, wherein the oxygen carrying solution is effective for prevention of entry of digestive enzymes into a wall of the intestine.

3. The method of claim 2, wherein the oxygen carrying solution includes perfluorocarbon.

4. The method of claim 3, wherein the oxygen carrying solution includes Perfluorodecalin.

5. The method of claim 4, wherein the oxygen carrying solution includes an ATP solution.

6. The method of claim 5, wherein the oxygen carrying solution includes ATP-MgCl₂.

7. The method of claim 5, wherein the oxygen carrying solution includes a buffer solution.

8. The method of claim 7, wherein buffer solution comprises HEPES.

9. The method of claim 1, wherein the oxygen carrying solution can be administered into a lumen of the intestine orally, by nasogastric (NG) tube into a stomach, or by catheter into a duodenum before or during intestinal ischemia.

10. A method for treating hypoxic breakdown of a mucosal barrier, the method comprising:
administering to an individual a therapeutic dose of an enzyme prohibiting solution.

11. The method of claim 10, wherein the enzyme prohibiting solution is effective for prevention of entry of digestive enzymes into a wall of the intestine.

12. The method of claim 11, wherein the enzyme includes trypsin.

13. The method of claim 12, wherein the enzyme prohibiting solution includes an oxygen carrying solution.

14. The method of claim 13, wherein the enzyme prohibiting solution includes perfluorocarbon.

15. The method of claim 14, wherein the enzyme prohibiting solution includes Perfluorodecalin.

16. The method of claim 15, wherein the oxygen carrying solution includes an ATP solution.

17. The method of claim 16, wherein the enzyme prohibiting solution includes ATP-MgCl₂.

18. The method of claim 16, wherein the enzyme prohibiting solution includes a buffer solution.

19. The method of claim 18, wherein buffer solution comprises HEPES.

20. The method of claim 10, wherein the oxygen carrying solution can be administered into a lumen of the intestine orally, by nasogastric (NG) tube into a stomach, or by catheter into a duodenum before or during intestinal ischemia.
